SEQUENCE LISTING (6 sequences)

SEQ ID NO: 1 - DNA, 21 bp, Artificial Sequence
  Col1a1_F primer
gagggccaagacgaagacatc

SEQ ID NO: 2 - DNA, 21 bp, Artificial Sequence
  Col1a1_R primer
cagatcacgtcatcgcacaac

SEQ ID NO: 3 - DNA, 20 bp, Artificial Sequence
  Col3a1_F primer
gttttgccccgtattatgga

SEQ ID NO: 4 - DNA, 20 bp, Artificial Sequence
  Col3a1_R primer
ggaagttcaggattgccgta

SEQ ID NO: 5 - DNA, 21 bp, Artificial Sequence
  Beta-actin_F primer
ggattcctatgtgggcgacga

SEQ ID NO: 6 - DNA, 21 bp, Artificial Sequence
  Beta-actin_R primer
cgctcggtgaggatcttcatg

(19) United States
(12) Patent Application Publication
(10) Pub. No.: US 2022/0193147 A1
(43) Pub. Date: Jun. 23, 2022

(54) Anti-aging Composition Containing Akkermansia Muciniphila as Active Ingredient and a Method for Preventing Aging Using Thereof

(71) Applicant: KOREA RESEARCH INSTITUTE OF BIOSCIENCE AND BIOTECHNOLOGY, Daejeon (KR)

(72) Inventors: Chul-Ho LEE, Daejeon (KR); Byoung-chan Kim, Daejeon (KR); Yong-Hoon Kim, Daejeon (KR); Jung-Ran Noh, Daejeon (KR); Jae-Hoon Kim, Daejeon (KR); Kyoung-Shim Kim, Daejeon (KR); Dong-Hee Choi, Daejeon (KR); Young-Keun Choi, Daejeon (KR); Dong-Ho Chang, Daejeon (KR); Haiyoung Jung, Daejeon (KR); Jung Hwan Hwang, Daejeon (KR)

(21) Appl. No.: 17/512,813

(22) Filed: Oct. 28, 2021

Related U.S. Application Data
(63) Continuation-in-part of application No. PCT/KR2019/012024, filed on Sep. 18, 2019.

(30) Foreign Application Priority Data
Oct. 1, 2018 (KR) 10-2018-0117123

(51) Int. Cl. A61K 35/74; C12N 1/20

(52) U.S. Cl. CPC A61K 35/74; C12N 1/20

(57) ABSTRACT

The present invention relates to a composition containing as an active ingredient at least one selected from the group consisting of Akkermansia muciniphila cells, a culture thereof, a lysate thereof, and an extract of the lysate or culture, and to an anti-aging method including administering the composition.

### TECHNICAL FIELD

The present invention relates to an anti-aging composition containing as an active ingredient at least one selected from the group consisting of Akkermansia muciniphila cells, a culture thereof, a lysate thereof, and an extract of the lysate or culture, and an anti-aging method including administering the composition.

### BACKGROUND ART

Humans are facing various problems not seen before due to the advent of an aging society caused by a prolonged average human lifespan. In socio-economic aspects, the elderly sustenance allowance per head is expected to increase due to an increase in the elderly population and a reduction in the productive age population, and there is also a growing interest in the improvement of the quality of life of the elderly. As the social demand for healthy and happy lives for the elderly increase as described above, studies on changes in the aspect of diseases resulting from aging and the prevention of aging-related diseases are being actively conducted.

The aging process causes a wide variety of changes. There are various internal changes, such as reduced functions of respective main tissues, food intake and digestive disorders, reduced brain function including defective memory, and reduced cardiovascular function, as well as various external changes, such as skin wrinkles, hair discoloration, curvature of the spine, and changes in movement. Moreover, these changes induce the reduction of function and cause diseases of the respective tissues, and therefore, it is very important to understand the causes of decline in external and internal functions due to aging and develop techniques for regulating these functions.

Among studies on aging, the fields that have received attention are lifespan control with respect to aging or functional recovery against aging. Studies on the extension of lifespan through various methods are rapidly increasing in recent years, such as extending lifespan by inhibiting the expression of a particular gene or overexpressing the particular gene in studies using drosophila models or nematodes, extending lifespan through diet restriction, and extending lifespan by treatment with rapamycin or the like (Nature Reviews Neuroscience volume 12, pages 437-452 (2011)). In addition, interest in the maintenance of function or recovery of function, instead of the simple extension of lifespan, is also a growing trend. However, regulation of a particular gene with reference to results shown in lower animal models may cause other functional side effects, and thus has a limitation in its application to humans. Moreover, treatment with a drug, such as rapamycin, may greatly influence immune function.

Korean Patent No. 10-1476236 discloses “lactic acid bacteria having activity to prevent and/or treat aging and dementia”, and Korean Patent Publication No. 2015-0093711 discloses “use of Akkermansia for treating a metabolic disorder”, but anti-aging effects of Akkermansia muciniphila have not been known.

### DISCLOSURE

### Technical Problem

The present inventors conducted intensive research efforts to prevent aging by using a substance non-toxic to the human body even when taken, as a safe medicine capable of effectively inhibiting and ameliorating aging and having no side effects for treating aging-related diseases, and as a result, identified that Akkermansia muciniphila showed effects of inhibiting and mitigating aging when administered to animal models, thereby completing the present invention.

### Technical Solution

An object of the present invention is to provide a composition containing as an active ingredient at least one selected from the group consisting of Akkermansia muciniphila cells, a culture thereof, a lysate thereof, and an extract of the lysate or culture.

Another object of the present invention is to provide a method for preventing or ameliorating aging, the method including administering the pharmaceutical composition to a non-human subject.

### Advantageous Effects

The anti-aging compositions containing as an active ingredient at least one selected from the group consisting of Akkermansia muciniphila cells, a culture thereof, a lysate thereof, and an extract of the lysate or culture of the present invention have effects of inhibiting weakness in muscular strength and change in hematopoietic stem cell compositions, and thus can effectively prevent and treat various aging symptoms.

### BRIEF DESCRIPTION OF DRAWINGS

In the drawing of the present invention, Vehicle represents a control group, AK represents a live Akkermansia strain administration group, and AK-P represents a dead Akkermansia strain administration group.

FIG. 1 shows frailty scores of aged mice administered with the live or dead Akkermansia strain.

FIG. 2 shows the measurement of muscle strength of aged mice administered with the live or dead Akkermansia strain, by using a grip strength meter.

FIG. 3 shows the muscle weight relative to body weight of aged mice administered with the live or dead Akkermansia strain.

FIG. 4 identifies the size of muscle fibers of aged mice administered with the live or dead Akkermansia strain. FIG. 4A shows immunostaining images for laminin; FIG. 4B shows the number of muscle fibers according to the size of the tibialis anterior (TA) muscle; and FIG. 4C shows the mean size of all of the muscle fibers.

FIG. 5 shows the qRT-PCR comparison in the mRNA expression levels of myogenin (Myog) and myosin heavy chain (MyHC) when C2C12 skeletal muscle myoblasts were treated with the live or dead Akkermansia strain.

FIG. 6 shows the numbers of LT-HSCs, ST-HSCs, and MPPs as percentages by measuring, by flow cytometry, the hematopoietic stem cell composition of aged mice administered with the live or dead Akkermansia strain.

FIG. 7 shows the measurement of the proportion of neutrophils and lymphocytes in the peripheral blood of aged mice administered with the live or dead Akkermansia strain.

FIG. 8 shows the results of analyzing the expressions of collagen Col1a1 and Col3a1 genes inhibiting skin wrinkles after human skin keratinocytes (HaCaT) were irradiated with UVB to induce photo-aging and treated with an AK lysate.

FIG. 9 shows the results of western blot and quantification analysis on the intracellular expression levels of MMP-1 and MMP-3 proteins that degrade collagen proteins after human skin keratinocytes (HaCaT) were irradiated with UVB to induce photo-aging and treated with an AK lysate.

FIG. 10 shows the results of comparative analysis of the amount of secretion of type I procollagen after the photo-aging-induced models obtained by irradiation of human fibroblasts with UVB were treated with an AK lysate.

### DETAILED DESCRIPTION OF THE INVENTION

The present invention will be specifically described as follows. Each description and exemplary embodiment disclosed in this invention may also be applied to other descriptions and exemplary embodiments. That is, all combinations of various elements disclosed in this invention fall within the scope of the present invention. In addition, the scope of the present invention is not limited by the specific description below.

Also, a person skilled in the art could recognize or identify numerous equivalents with respect to certain aspects of the present invention only by routine experiments. Furthermore, such equivalents are intended to be encompassed by the present invention.

In accordance with an aspect of the present invention to attain the objects, there is provided an anti-aging composition containing as an active ingredient at least one selected from the group consisting of Akkermansia muciniphila cells, a culture thereof, a lysate thereof, and an extract of the lysate or culture.

“Akkermansia muciniphila” of the present invention is a gram-negative, strictly anaerobic, non-motile, non-spore-forming, oval-shaped bacterium. The Akkermansia muciniphila bacteria use mucin as sole source of carbon and nitrogen thereof, and are known to inhabit the gastrointestinal tract of various animals including humans.

Specifically, Akkermansia muciniphila of the present invention may be strains deposited at the American Type Culture Collection under accession number ATCC BAA-835 and at the German Collection of Microorganisms and Cell Cultures under accession number DSM 22959, but could include any strain without limitation so long as the strain has an anti-aging effect. In addition, cells of the Akkermansia muciniphila strain, a culture of the strain, a lysate of the strain, and an extract of the lysate or culture may also be included in the scope of the present invention.

The Akkermansia muciniphila of the present invention includes both live and dead cell forms. For example, the Akkermansia muciniphila of the present invention may be used in the form of live cells, dead cells, or a mixture of live and dead cells. For example, Akkermansia muciniphila may exist in a dried, lyophilized, or heated form. However, Akkermansia muciniphila may be used in the form suitable for inclusion in various compositions, without the limitation to the above-described examples.

The term “aging” of the present invention includes a concept encompassing degenerative changes that occur due to the deterioration of body structures and functions with age, and changes due to aging are very diverse, such as reductions in each tissue weight and body weight resulting from a decrease in the number of parenchymal cells, changes in connective tissues, a change in body composition, a reduction in elasticity of blood vessels or skin, a decline in each organ function, a reduction in disease recovery ability including immunity, declines in sensory organ functions, and deteriorations in memory, learning ability, and comparison ability. However, degenerative brain diseases including deteriorations in cognitive function and memory, Parkinson's disease, and dementia have recently been lowered in age of onset and may occur even in patients of lower age, and thus are not included in the concept of “aging” of the present invention.

Specifically, for the purpose of the present invention, the aging may include at least one aging from the group consisting of muscle aging, skin aging, vision aging, hearing aging, digestive organ aging, immunosenescence, and urinary organ aging.

The “muscle aging” of the present invention is used interchangeably with “muscle senescence”, and includes a concept encompassing muscular decline that accompanies aging, for example, deterioration in muscular functions (muscular strength, muscular endurance, instantaneous muscular power, etc.), muscular atrophy, and the like. The muscular atrophy refers to a reduction in muscle mass due to the reduction or contraction of muscle cells. The muscle aging may gradually degrade muscle density and muscular functions after the age of 30 and may easily cause falls and fractures. The causes of muscle aging may be reduced growth hormones and male hormones, reduced ability to synthesize protein in the body, weakened ability to absorb proteins or calories associated with the maintenance of muscle density, and the like.

The “skin aging” of the present invention includes symptoms of reduced elasticity, luster loss, wrinkle formation, weakened regenerative capability, severe dryness, and the like in the skin, and may be caused by the passage of time, external environments, and the like. The skin aging includes both intrinsic aging that naturally occurs with the passage of time and photo-aging that occurs in the skin due to the ultraviolet light. The skin aging of the present invention may result in reductions in synthesis amounts of collagen, hyaluronic acid, elastin, proteoglycan, fibronectin and/or precursors thereof, increases in expressions of degrading enzymes of the components, reductions in expressions of synthesizing enzymes of the components, or the like in the skin cells.

The “vision aging” of the present invention refers to reduced vision accompanying aging due to various causes, such as, a deterioration in lens accommodation resulting from a reduction in lens elasticity with age, a reduction in ciliary muscle fiber elasticity, and cornea sclerosis, and may include symptoms commonly referred to as presbyopia and the resultant diseases, such as dry eye syndrome, macular degeneration, hyperopia, myopia, and cataracts.

The “hearing aging” of the present invention refers to a gradual loss of hearing accompanying aging, and may be caused by reductions in the number of neurons connected to the inner ear, middle ear, and brain and declines in functions thereof, and may be accompanied by tinnitus, hearing loss, or the like.

The “digestive organ aging” of the present invention refers to changes in the oral cavity, esophagus, and gastrointestinal system, and may be accompanied by symptoms, for example, reduced gastric acid secretion and pancreatic juice secretion, reduced rate and efficiency of digestion resulting from reduced motility of the gastrointestinal tract, or significantly reduced absorption rate of ingested nutrients, such as a fat, resulting in dyspepsia, diarrhea, and the like.

The term “immunosenescence” of the present invention refers to changes in immune functions (Miller R. A. Science 1996; 273, 70, Won D. I. et al., Korean J Lab Med 2003; 23, 205, and Ben-Yehunda et al., Cancer Invest 1992; 10, 525), and examples thereof may be an increased number of neutrophils and a decreased number of lymphocytes. Lymphocytes are a type of white blood cells that are produced by the differentiation and maturation of hematopoietic stem cells as progenitor cells into lymphocyte-based hematopoietic stem cells through the hematopoietic process. Since the lymphocytes are involved in a specific immune response, a decrease in the number of lymphocytes may be one factor that causes a deterioration in immunity. However, the relationship between immunosenescence and the deterioration in immunity is not limited thereto. The immunosenescence may cause diseases, such as autoimmune disease, pneumonia, flu, tetanus, infectious endocarditis, and cancer, or may accelerate the worsening of the disease symptoms, but is not limited thereto. The diseases may be caused by aging.

The “urinary organ aging” of the present invention includes symptoms caused by changes with age in intra-abdominal pressure, muscular strength of the pelvis, urethra, and bladder, and thickening of urethral mucosa, and may be accompanied by diseases, such as overactive bladder, urinary incontinence, enlarged prostate, lower urinary tract symptoms, glomerulonephritis, and chronic renal failure.

In an embodiment of the present invention, the inhibition of aging was identified when Akkermansia was administered to aged mouse models and visual examination was performed on the skin, musculoskeletal system, auditory system, visual/olfactory system, digestive/urogenital system, and the like. It can therefore be seen that Akkermansia has an anti-aging effect.

The “anti-aging” of the present invention refers to any action that inhibits, suppresses, or delays the above-described aging symptoms due to the administration of the composition of the present invention, and specifically means any action that at least reduces parameters associated with the above-described aging, for example, severity of a symptom, and encompasses any action that alleviates, relieves, or favorably changes aging symptoms due to the administration of the composition of the present invention. In addition, the term may be used interchangeably with “inhibition of aging”.

For the purpose of the present invention, the composition may be characterized by any one of inhibition of muscular strength weakness, inhibition of hematopoietic stem cell aging, inhibition of immunosenescence, promotion of myoblast differentiation, and inhibition of skin aging.

Specifically, the term “inhibition of muscular strength weakness” may refer to inhibiting the deteriorations in muscular strength, muscular endurance, instantaneous muscular power, and the like, which are the above-described symptoms of muscle aging, or a reduction in muscle mass due to a reduction or contraction of muscle cells, and may be evaluated through a muscular function test that measures temporary maximum muscular strength, such as grip strength, back muscular strength, arm muscular strength, or leg muscular strength, or muscular endurance that enables the repetition of exercise with a predetermined load.

In an embodiment of the present invention, it was tested whether Akkermansia had an effect of inhibiting muscular strength weakness, by measuring the maximum grip strength of mice after the administration of Akkermansia to aged mice, and as a result, it was identified that the muscular strength was increased at 8 weeks and 16 weeks of Akkermansia administration. In another embodiment of the present invention, Akkermansia was administered to aged mice, and the muscle weight and muscle fiber size were measured, and as a result, the Akkermansia administration groups were identified as showing increases in muscle weight and increases in muscle fiber size. It can therefore be seen that Akkermansia has an effect of inhibiting muscular strength weakness.

The term “hematopoietic stem cell (HSC)” of the present invention refers to a representative adult stem cell that can be differentiated into all types of blood cells to provide blood cells during the lifetime. Hematopoietic stem cells produce blood during the lifetime and show high turnover. In the distribution of marrow hematopoietic stem cells (HSCs), the proportion of long-term HSCs (LT-HSCs) tends to increase, and the proportion of multipotent progenitors (MPPs) tends to decrease during aging. The aging of hematopoietic stem cells results in declines in immune functions and develops aging-related diseases, and thus the aging of hematopoietic stem cells may be a cause of widespread aging of body organs.

In an embodiment of the invention, it was identified that the administration of Akkermansia muciniphila to aged mice resulted in a change in the composition of hematopoietic stem cells, a significant reduction in LT-HSCs, and a significant increase in MPPs. It can therefore be seen that Akkermansia has an effect of inhibiting the aging of hematopoietic stem cells.

The term “inhibition of immunosenescence” refers to the suppression, treatment, and/or alleviation of the above-described symptoms of immunosenescence and symptoms of diseases developed and aggravated as a result thereof.

In an embodiment of the present invention, it was identified that the administration of Akkermansia muciniphila to aged mice resulted in a comparative decrease in the number of neutrophils and an increase in the number of lymphocytes, and it can therefore be seen that Akkermansia muciniphila is effective in the treatment and alleviation of immunity deterioration and immune diseases caused by aging.

The term “myoblast” of the present invention refers to a muscle cell in an undifferentiated state, and the differentiation of myoblasts into skeletal muscle cells forms a muscle tissue, so the differentiation of myoblasts is also referred to as myogenesis. The factors involved in such myoblast differentiation include Mef2, serum response factor (SRF), MyoD, Myf5, Myf6, myogenin, myosin heavy chain, and the like, and the differentiation of myoblasts may be determined by the measurement of expression levels of these factors.

In an embodiment of the present invention, myoblasts were cultured by treating skeletal muscle myoblasts with Akkermansia, and then the mRNA expression levels of myogenin and myosin heavy chain, which are representative factors involved in skeletal muscle differentiation, were measured. As a result, it was identified that the treatment with Akkermansia resulted in significant increases in expression of myogenin and myosin heavy chain. It can therefore be seen that Akkermansia has effects of promoting and improving the differentiation of skeletal muscle myoblasts, and furthermore, it would be obvious that the promotion of myoblast differentiation can inhibit or ameliorate muscle aging.

The “inhibition of skin aging” of the present invention may encompass an increase in the amount of synthesis of collagen or a precursor thereof in skin cells. The skin cells include skin keratinocytes and skin fibroblasts.

In an embodiment, the inhibition of skin aging of the present invention may encompass an increase in collagen synthetase activity and/or an inhibition of collagenase activity. Examples of the collagen synthetase may include Col1a1 and/or Col3a1. Examples of the collagenase may include MMP-1 and/or MMP-3. However, the examples are not limited thereto.

In an embodiment, the inhibition of skin aging of the present invention may encompass an improvement in skin tone, a relief in skin wrinkles, and/or an enhancement in skin elasticity. However, the inhibition of skin aging is not limited thereto.

In an embodiment of the present invention, human skin cells were treated with Akkermansia muciniphila to measure the expression levels of collagen synthesis and degradation genes, and as a result, it was identified that the expression of collagen synthesis genes was increased, the expression of collagen degradation genes was inhibited, and the amount of secretion of Type 1 procollagen, a precursor of collagen, was significantly increased. It can therefore be seen that Akkermansia has a skin aging inhibitory effect.

Through the above-described test results, it can be seen that the composition containing as an active ingredient at least one selected from the group consisting of Akkermansia muciniphila cells, a culture thereof, a lysate thereof, and an extract of the lysate or culture of the present invention has effects of inhibiting the reduction in elasticity of blood vessels or skin, deterioration in immunity, decline in each organ function, and muscle aging and skin aging.

The contents of the Akkermansia muciniphila cells, the culture thereof, the lysate thereof, and the extract of the lysate or culture contained in the composition of the present invention are not limited as long as the composition has an anti-aging effect, but these may be contained in a content of 0.0001 wt % to 99.9 wt %, and more specifically 0.01 wt % to 80 wt % relative to the total weight of the final composition.

In an embodiment, the composition of the present invention may be a pharmaceutical composition.

The pharmaceutical composition of the present invention may further contain an appropriate carrier, excipient, or diluent that is commonly used in the preparation of a pharmaceutical composition. As used herein, the term “pharmaceutically acceptable carrier” refers to a carrier or a diluent that does not inhibit biological activity and characteristics of a compound to be administered, while causing no stimulation to an organism.

The carrier usable in the present invention is not particularly limited to the kind thereof, and any carrier may be used as long as the carrier is commonly used in the art and is pharmaceutically acceptable. Non-limiting examples of the carrier may include a saline solution, sterile water, Ringer's solution, buffered physiological saline, an albumin infusion solution, a dextrose solution, a maltodextrin solution, glycerol, ethanol, and the like. These may be used alone or in a mixture of two or more thereof.

In addition, the composition of the present invention may be used by addition of other common additives, such as an antioxidant, a buffer and/or a bacteriostatic agent, as needed, and may be formulated into injectable formulations, such as an aqueous solution, a suspension, and an emulsion, pills, capsules, granules, tablets, and the like, by addition of a diluent, a dispersant, a surfactant, a binder, and/or a lubricant. The pharmaceutical composition of the present invention may be manufactured in various formulations according to whether the desired manner of administration is oral administration or parenteral administration.

Non-limiting examples of the formulation for oral administration may include troches, lozenges, tablets, water-soluble suspensions, oily suspensions, formulated powders, granules, emulsions, hard capsules, soft capsules, syrups, elixirs, or the like.

In order to prepare the composition of the present invention into formulations for oral administration, such as tablets or capsules, the composition may contain: a binder, such as lactose, saccharose, sorbitol, mannitol, starch, amylopectin, cellulose, or gelatin; an excipient, such as dicalcium phosphate; a disintegrant, such as corn starch or sweet potato starch; and a lubricant, such as magnesium stearate, calcium stearate, sodium stearyl fumarate, and polyethylene glycol wax. Furthermore, the composition of the present invention, for a capsule formulation, may further contain a liquid carrier, such as a fatty oil, in addition to the aforementioned materials.

For formulations for parenteral administration, the composition of the present invention may be prepared into, for example, a form for injection, such as subcutaneous injection, intravenous injection, or intramuscular injection; and a suppository injectable form; a form for spraying, such as an aerosol, so as to permit inhalation through a respirator, but are not limited thereto. For the preparation into a formulation for injection, the composition of the present invention may be mixed with a stabilizer or a buffer in water to prepare a solution or a suspension, which is then prepared in a unit dose of an ampoule or vial. When the composition is formulated into a spray, such as an aerosol, a propellant or the like may be mixed with an additive so that a water-dispersed concentrate or a wet powder is dispersed.

The pharmaceutical composition of the present invention may be administered in a pharmaceutically effective amount. The term “pharmaceutically effective amount” of the present invention refers to an amount sufficient for the treatment or prevention of a disease at a reasonable benefit/risk ratio applicable to a medical treatment or prevention, and the level of effective dose may be determined according to: the factors including severity of illness, drug activity, a patient's age, body weight, health, and sex, drug sensitivity of a patient, administration time, administration route, excretion rate, and length of treatment of the composition used in the present invention, and a drug to be mixed or concurrently used in combination with the composition used in the present invention; and other factors well known in the medical field.

The pharmaceutical composition of the present invention may be administered as an individual treatment or in combination with another treatment, and may be administered sequentially or simultaneously with conventional treatments. In addition, the pharmaceutical composition may be administered once or multiple times. The pharmaceutical composition may be administered in an amount at which a maximum effect can be attained with a minimum amount without side effects.

As for the dose of the pharmaceutical composition of the present invention, the pharmaceutical composition of the present invention may be administered to animals including humans at 0.1 mg/kg to 500 mg/kg of body weight per day, but is not limited thereto. The administration frequency of the composition of the present invention may be once or several times using divided doses per day, but is not particularly limited thereto. The above dose is not intended to limit the scope of the present invention in any aspect.

In an embodiment, the composition of the present invention may be a quasi-drug composition.

The term “quasi-drug” of the present invention refers to a product that is not an instrument, machine, or device, among the products used for diagnosing, curing, relieving, treating, preventing, or alleviating diseases of humans or animals, and to a product that is not an instrument, machine, or device, among the products used for exerting pharmaceutical influences on structures and functions of humans or animals, and also encompasses externally applied preparations for skin and personal hygiene products.

The externally applied preparations for skin may be specifically prepared and used in the form of an ointment, a lotion, a spray, a patch, a cream, a powder, a dispersion, a gelling agent, or a gel, but are not particularly limited thereto. Examples of the personal hygiene products may include soaps, cosmetics, wet tissues, toilet paper rolls, shampoos, skin creams, facial creams, toothpastes, lipsticks, perfumes, make-ups, foundations, blushers, mascaras, eye shadows, sunscreen lotions, hair care products, air-freshener gels, or cleansing gels. In addition, other examples of the quasi-drug composition of the present invention may include disinfectants, shower foams, mouthwashes, wet tissues, detergents, hand washes, humidifier fillers, masks, or ointments.

In an embodiment, the composition of the present invention may be a cosmetic composition.

The cosmetic composition of the present invention may be prepared in the formulation selected from the group consisting of solutions, externally applied ointments, creams, foams, nutritious skin lotions, softening skin lotions, packs, softeners, milky liquids, makeup bases, essences, soaps, liquid soap materials, bath preparations, sun screen-creams, sun oils, suspensions, emulsions, pastes, gels, lotions, powders, surfactant-containing cleansing agents, oils, powder foundations, emulsion foundations, wax foundations, patches, and sprays, but is not limited thereto.

In addition, the cosmetic composition of the present invention may further contain at least one cosmetically acceptable carrier that is mixed with typical skin cosmetic materials. For example, typical components, such as oil components, water, surfactants, moisturizers, lower alcohols, thickeners, chelating agents, dyes, preservatives, and fragrances, may be appropriately mixed, but are not limited thereto.

The cosmetically acceptable carrier contained in the cosmetic composition of the present invention may include a suitable material according to the formulation.

In accordance with another aspect of the present invention, there is provided a method for preventing or ameliorating aging, the method including administering, to a non-human subject, a composition containing as an active ingredient at least one selected from the group consisting of Akkermansia muciniphila cells, a culture thereof, a lysate thereof, and an extract of the lysate or culture.

As described above, the Akkermansia muciniphila provided in the present invention has an effect of preventing or ameliorating aging, and thus the composition containing the same can be used to prevent or ameliorate aging.

The “subject” of the present invention may refer to any animal including humans. The animal may be not only a human but also a mammal, such as a cow, a horse, a sheep, a pig, a goat, a camel, an antelope, a dog, or a cat, in need of treatment for a similar symptom to the human. The subject may refer to a non-human subject, but is not limited thereto.

The “administration” of the present invention refers to an introduction of the composition of the present invention into a subject by any suitable method. The composition of the present invention may be administered through various routes of oral or parenteral administration as long as the composition can reach a target tissue.

As for the administration route of, for example, a pharmaceutical composition, the pharmaceutical composition may be administered through any general route as long as the composition can reach a target tissue. The pharmaceutical composition of the present invention may be administered through a route, such as intraperitoneal administration, intravenous administration, intramuscular administration, subcutaneous administration, intradermal administration, oral administration, intranasal administration, intrapulmonary administration, or rectal administration, according to the desired purpose, but is not particularly limited thereto. However, the composition may be denatured by gastric acid during oral administration, and thus a composition for oral administration needs to be formulated such that an active drug is coated or protected from degradation in the stomach. The composition may also be administered by any device that can deliver an active substance to a target cell.

In accordance with another aspect of the present invention, there is provided an anti-aging health functional food composition containing as an active ingredient at least one selected from the group consisting of Akkermansia muciniphila cells, a culture thereof, a lysate thereof, and an extract of the lysate or culture.

The health functional food of the present invention may be manufactured by a method that is commonly used in the art, and may be manufactured by adding raw materials and ingredients that are conventionally added in the art. In addition, the health functional food may also be manufactured in any formulation without limitation as long as the formulation is acceptable as a food. The health functional food composition of the present invention may be prepared in various formulations, contains food as a raw material unlike general drugs and thus has an advantage of having no side effects that may occur in long-term use of a drug, can be usually ingested due to excellent portability and thus is very useful, and can be ingested as a supplement for enhancing effects of preventing or ameliorating aging.

The health functional food is not particularly limited in other ingredients, except for the Akkermansia muciniphila cells, the culture thereof, the lysate thereof, and the extract of the lysate or culture as essential ingredients, and may contain several herbal extracts, food supplement additives, or natural carbohydrates as additional ingredients, like in typical health functional foods. The food supplement additives include food supplement additives that are conventional in the art, for example, flavoring agents, savoring agents, coloring agents, fillers, stabilizers, and the like.

Examples of the natural carbohydrates may include ordinary sugars, for example, monosaccharides, such as glucose and fructose; disaccharides, such as maltose and sucrose, and polysaccharides, such as dextrin and cyclodextrin; and sugar alcohols, such as xylitol, sorbitol, and erythritol. In addition to the above-described additives, natural flavoring agents (e.g., rebaudioside A, glycyrrhizin, etc.) and synthetic flavoring agents (saccharin, aspartame, etc.) may be advantageously used as the flavoring agents.

Apart from the above ingredients, the health functional food composition of the present invention may contain various nutrients, vitamins, water (electrolytes), flavoring agents, such as synthetic flavoring agents and natural flavoring agents, coloring agents, extenders (cheese, chocolate, etc.), pectic acid and salts thereof, alginic acid and salts thereof, organic acids, protective colloidal thickeners, pH adjusters, stabilizers, preservatives, glycerin, alcohols, carbonating agents used for carbonated drink, and the like, and may also contain fruit flesh for manufacturing natural fruit juices, fruit juice drinks, and vegetable drinks. These ingredients may be used either alone or in combination. In addition, the health functional food may be in the form of any one of meat, sausage, bread, chocolate, candies, snacks, confectioneries, pizzas, ramen, other noodles, gums, ice creams, soups, drinking water, teas, functional water, drinks, alcoholic drinks, and vitamin complexes.

The health functional food of the present invention may further contain food additives, and the suitability thereof as a “food additive”, unless otherwise specified, is determined by the standards and criteria for the corresponding item in accordance with the General Rules and General Test Methods of the Food Additive Code approved by the Ministry of Food and Drug Safety.

The content of the composition that is added to food including drinks, in the process of manufacturing a health functional food, may be appropriately increased or reduced as needed.

In accordance with another aspect of the present invention, there is provided an anti-aging feed additive composition containing as an active ingredient at least one selected from the group consisting of Akkermansia muciniphila cells, a culture thereof, a lysate thereof, and an extract of the lysate or culture.

The feed composition may contain a feed additive.

The term “feed additive” in the present invention includes substances that are added to feed for the purpose of various effects, such as supplementing nutrients, preventing weight loss, promoting digestibility of cellulose in the feed, improving milk quality, preventing reproductive disorders, improving a rate of pregnancy, and preventing high-temperature stress during the summer season. The feed additive of the present invention may correspond to supplementary feed according to the Control of Livestock and Fish Feed Act.

The term “feed” in the present invention refers to any natural or artificial diet, a single meal, or the like, or an ingredient of the single meal, which an animal eats, ingests, and digests or which is suitable for eating, ingestion, and digestion. The feed containing the anti-aging composition according to the present invention as an active ingredient may be prepared in various forms of feed known in the art.

The type of feed is not particularly limited, and feeds commonly used in the art may be used. Non-limiting examples of the feeds may include: vegetable feeds, such as grains, root vegetables, food processing byproducts, algae, fibers, pharmaceutical byproducts, oils and fats, starches, residues, or grain byproducts; and animal feeds, such as proteins, inorganic materials, oils and fats, minerals, oils and fats, single-cell proteins, and animal planktons or foods. These may be used alone or in a mixture of two or more thereof.

The contents of the Akkermansia muciniphila cells, the culture thereof, the lysate thereof, and the extract of the lysate or culture in the feed composition of the present invention may be appropriately adjusted according to the type and age of applied livestock, type of application, and desired effect.

In the present invention, the above-described “anti-aging” may also be expressed as “treating of aging”, and also includes treatment and/or alleviation of aging-related diseases.

In accordance with another aspect of the present invention, there is provided a pharmaceutical composition for preventing, treating, or alleviating an aging-related disease, the pharmaceutical composition containing as an active ingredient at least one selected from the group consisting of Akkermansia muciniphila cells, a culture thereof, a lysate thereof, and an extract of the lysate or culture.

In accordance with another aspect of the present invention, there is provided an animal medicine for preventing, treating, or alleviating an aging-related disease, the animal medicine containing as an active ingredient at least one selected from the group consisting of Akkermansia muciniphila cells, a culture thereof, a lysate thereof, and an extract of the lysate or culture. In accordance with another aspect of the present invention, there is provided a method for preventing, treating, or alleviating an aging-related disease, the method including administering a composition containing as an active ingredient at least one selected from the group consisting of Akkermansia muciniphila cells, a culture thereof, a lysate thereof, and an extract of the lysate or culture.

The aging-related disease may be a disease caused by at least one aging from the group consisting of muscle aging, skin aging, vision aging, hearing aging, digestive organ aging, immunosenescence, and urinary organ aging.

Examples of the aging-related disease may be at least one disease selected from sarcopenia, photo-aging, dry eczema, skin pigmentation, solar lentigo, seborrheic keratosis, actinic keratosis, pruritus, impetigo, folliculitis, cellulitis, herpes zoster, dry eye syndrome, macular degeneration, hyperopia, myopia, cataract, tinnitus, deafness, dyspepsia, diarrhea, autoimmune disease, pneumonia, flu, tetanus, Infectious endocarditis, skin cancer, cancer, overactive bladder, urinary incontinence, prostatic hyperplasia, lower urinary tract symptoms, glomerulonephritis, and chronic renal failure, but are not limited thereto, and may include any disease that is caused by aging, without limitation.

### MODE FOR CARRYING OUT THE INVENTION

Hereinafter, the present disclosure will be described in detail with reference to examples and experimental examples. However, these examples and experimental examples are provided for specifically illustrating the present invention, and the scope of the present invention is not limited to these examples and experimental examples.

### Example 1: Aged Animal Models and Akkermansia Strain Administration Method

The Akkermansia muciniphila strain used in the test was an Akkermansia muciniphila standard strain (AK; American Type Culture Collection accession number ATCC BAA-835, identical to DSM 22959), and this strain was purchased from ATCC and used in the present invention.

The 57BL/6 male mice aged 100 weeks were used as aged animal models. In Examples 2 to 8 using mice, the mice were divided into a vehicle group (control group) administered with only BTTM broth used for Akkermansia culture, an AK group in which a live Akkermansia muciniphila strain cultured in BTTM broth was administered at 3×10⁸ cells, and an AK-P group in which a dead strain obtained by heating the cultured Akkermansia strain at 70° C. for 30 minutes was administered. The administration for each group was conducted orally once a day for 20 weeks.

### Example 2: Visual Analysis of Frailty

To analyze the overall effect of Akkermansia strain administration on aging, visual examination was performed.

Specifically, the animal models and the strain administration method in Example 1 were used, and each individual mouse was subjected to visual examination at 0 and 16 weeks of administration for 25 items in 6 categories including the integument, musculoskeletal system, auditory system, visual/olfactory systems, digestive/urogenital system, and respiratory system. A score of 0 was given for no unusual symptoms, a score of 0.5 for usual symptoms, and a score of 1 for severe symptoms. The mean value as frailty index (FI) was calculated by adding up all of the scores and dividing by the number of aged mice in each group, and then comparative analysis was performed.

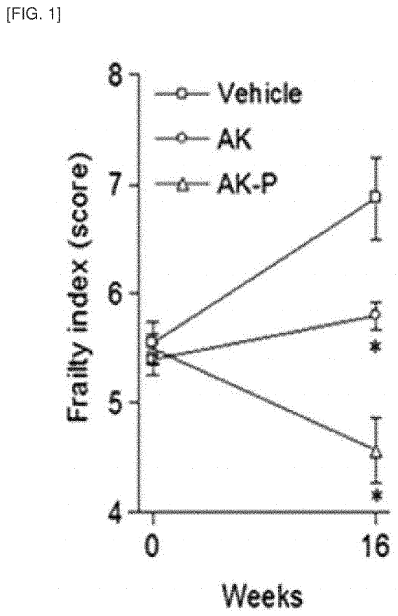

The results identified that at 0 weeks of administration, the FI values of the vehicle, AK, and AK-P groups were 5.6±0.2, 5.4±0.2, and 5.5±0.1, respectively, showing no significant difference, but at 16 weeks of administration, the FI of the vehicle group was 6.9±0.4 with an increase of about 1.3; the FI of the AK administration group was 5.8±0.1 with an increase of 0.4, indicating a relatively small increase; and the FI of the AK-P group was 4.6±0.3 with rather a decrease of about 0.9 (FIG. 1).

It can therefore be seen from the results that the administration of either the live or dead Akkermansia strain inhibits or ameliorates the progression of aging.

### Example 3: Analysis of Muscular Strength Aging

To analyze the effect of Akkermansia strain administration on muscular strength, the grip strength test was performed on aged mice.

Specifically, the animal models and the strain administration method in Example 1 were used. Each aged mouse was allowed to hold a wire mesh, connected to a muscular strength probe of a grip strength meter, with four limbs, and then the tail was carefully pulled backward to measure the maximum grip strength at the moment when the mouse gripped the wire mesh. The grip strength test was performed using the grip strength meter at 8 weeks and 16 weeks of strain administration.

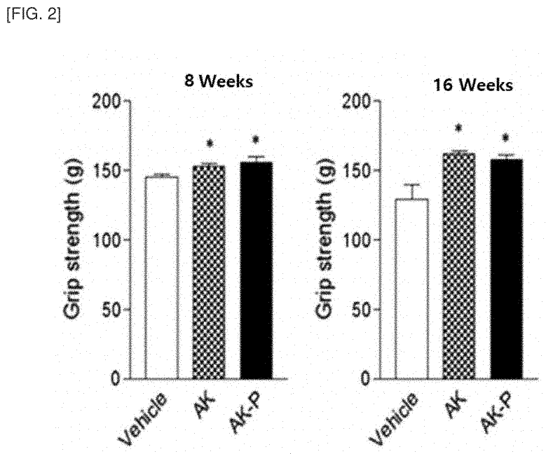

The results identified that at 8 weeks of strain administration, the grip strength of the vehicle control group was 145±2.2 (g), and in comparison, the grip strengths of the live Akkermansia strain administration group (AK group) and the dead Akkermansia strain administration group (AK-P group) were 153±1.5 (g) and 156±4.2 (g), respectively, indicating increases in muscular strength. The results identified that at 16 weeks of strain administration, the grip strength was 130±10 (g) in the vehicle group, indicating a deterioration in muscular strength, but 162±2.0 (g) in the AK group and 157±3.7 (g) in the AK-P group, indicating increases in muscular strength (FIG. 2).

It could therefore be seen from the results that the administration of live or dead Akkermansia strain inhibits or ameliorates muscle aging.

### Example 4: Analysis of Muscle Mass

To analyze the effect of Akkermansia strain administration on muscle mass, the muscle mass relative to body weight was measured.

Specifically, aged mice were administered with the Akkermansia strain as described in Example 1, and the tibialis anterior (TA), gastrocnemius (GC), and soleus muscles were isolated from the left and right limbs of each mouse and weighed, and then converted into the muscle weight relative to the body weight (Table 1).

TABLE 1

| | TA muscle | GC muscle | Soleus muscle |
|---|---|---|---|
| Control | 3.03 ± 0.08 mg | 7.29 ± 0.14 mg | 0.52 ± 0.04 mg |
| AK group | 3.53 ± 0.07 mg | 8.07 ± 0.19 mg | 0.62 ± 0.01 mg |
| AK-P group | 3.42 ± 0.07 mg | 8.62 ± 0.30 mg | 0.63 ± 0.02 mg |

As a result, the TA muscle weighed 3.03±0.08 mg in the vehicle control group, 3.53±0.07 mg in the live Akkermansia strain administration group (AK group), and 3.42±0.07 mg in the dead Akkermansia strain administration group (AK-P), indicating that the administration of the live Akkermansia strain (AK group) or Akkermansia strain (AK-P) significantly increased muscle mass.

The GC muscle also weighed 7.29±0.14 mg in the vehicle control group, and 8.07±0.19 mg and 8.62±0.30 mg in the live Akkermansia strain administration group (AK group) and the dead Akkermansia strain administration group (AK-P group), respectively, indicating significant increases in muscle mass compared with the vehicle control group.

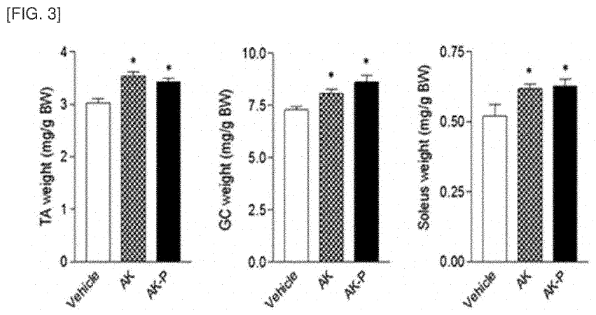

Similarly, the soleus muscle also weighed 0.52±0.04 mg in the vehicle control group, 0.62±0.01 mg in the live Akkermansia strain administration group (AK group), and 0.63±0.02 mg in the dead Akkermansia strain administration group (AK-P), indicating significant increases in muscle mass compared with the vehicle control group (FIG. 3).

Since an increase in muscle mass compared with the control group was shown for each of the three types of muscles, the Akkermansia strain was identified as significantly inhibiting the muscle mass reduction caused by aging.

### Example 5: Identification of Muscle Fiber Amount

To analyze the effect of Akkermansia strain administration on muscle fibers, the muscle fiber volume was measured.

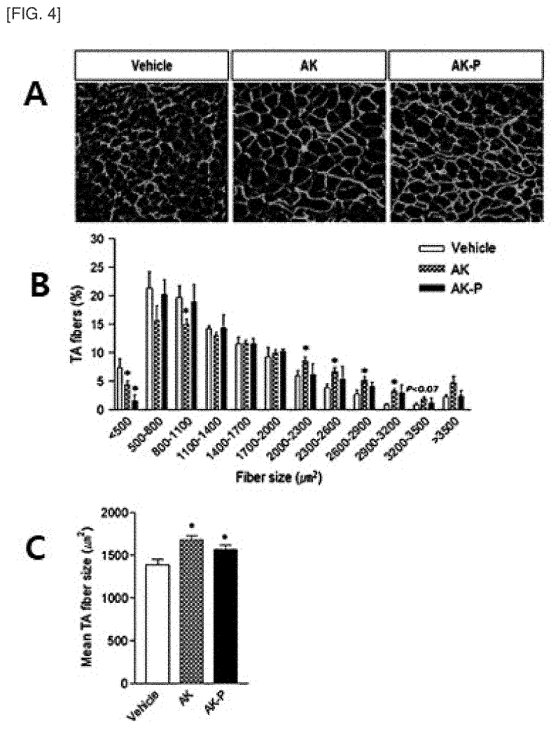

Specifically, aged mice were administered with the Akkermansia strain as described in Example 1, and the weight of left tibialis anterior (TA) muscle was measured. Thereafter, the muscle was fixed in 10% formalin and made into frozen sections and subjected to immunostaining for laminin, a main component of the muscle basement membrane. It was identified that strong fluorescence appeared in the Akkermansia strain administration groups (AK and AK-P) (FIG. 4A).

Next, confocal microscopy observation and muscle fiber imaging were performed, and the size distribution was obtained for respective cross-sectional sizes of muscle fibers from 500 μm² or smaller to 3500 μm² or larger by using an image analysis program, and the mean size of all of the muscle fibers was calculated.

The results identified that the live Akkermansia strain administration group (AK group) or the dead Akkermansia strain administration group (AK-P group), compared with the vehicle control group, showed significant decreases in the number of small-sized muscle fibers with a TA muscle fiber cross-section size of 500 μm² or smaller, but increases in the number of large-sized muscle fibers with a size of 2000 μm² or more (FIG. 4B).

Also, as a result of calculating the mean size of all of the muscle fibers including from small to large sizes, the mean size was 1,392.5±59.5 μm² in the vehicle control group, and 1,681.8±47.7 μm² and 1,567.1±50.3 μm² in the live Akkermansia strain administration group (AK group) or dead Akkermansia strain administration group (AK-P group), respectively, indicating that the mean size of all of the muscle fibers was significantly increased in the aged mice administered with the live Akkermansia strain (AK group) or dead Akkermansia strain (AK-P group) (FIG. 4C).

It was identified from the results that the Akkermansia strain has an effect of inhibiting muscle fiber atrophy caused by aging.

### Example 6: Analysis of Myoblast Differentiation Promoting Ability

To analyze the effect of Akkermansia strain administration on myoblasts, myoblasts were treated with the live or dead Akkermansia strain to measure the expression levels of myogenic regulatory factors.

Specifically, C2C12 skeletal muscle myoblasts were purchased from ATCC (USA) and cultured in DMEM containing 10% FBS, 100 U/mL penicillin, and 100 μg/mL streptomycin, at 37° C. under a 5% CO₂ condition. For the induction of differentiation of the cells, the cells were dispensed at 5×10⁵ cells/mL in 6-well plates, and when the cells grew to 90% or more, the medium was exchanged with a differentiation medium containing 2% horse serum, and the cells were cultured for 5 days. The live and dead Akkermansia strains were used after dilution to 1×10⁸ cells/mL in PBS. The medium was exchanged every two days, and the differentiation culture was ended after 5 days. Thereafter, the mRNA expression levels of myogenin (Myog) and myosin heavy chain (MyHC) were measured by qRT-PCR.

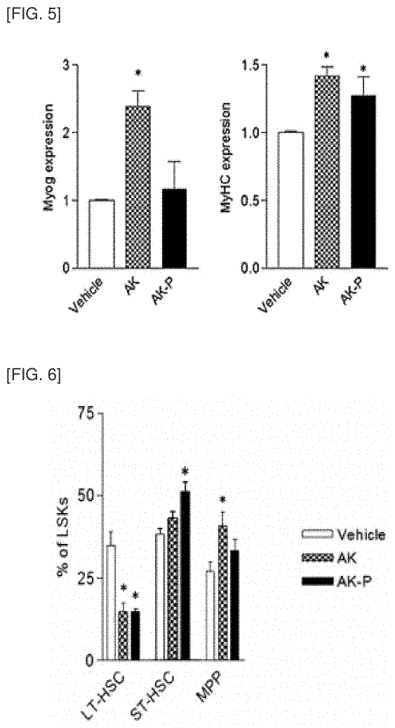

The results identified that the expressions of myogenin and myosin heavy chain were significantly increased in the administration of the live Akkermansia strain (AK group) and dead Akkermansia strain (AK-P group) compared with the vehicle control group (FIG. 5).

It was identified from the results that the Akkermansia strain has an effect of promoting or improving myoblast differentiation.

### Example 7: Analysis of Marrow Hematopoietic Stem Cells

To investigate the effect of Akkermansia strain administration on hematopoietic stem cells, the distributions of marrow hematopoietic stem cells of the mice administered with the Akkermansia strains were compared.

Specifically, in the distribution of marrow hematopoietic stem cells (HSCs), the proportion of long-term HSCs (LT-HSCs) tends to increase while the proportion of multipotent progenitors (MPPs) tends to decrease during aging. Based on this, the effect on the aging of hematopoietic stem cells was investigated through the comparison of the distribution of hematopoietic stem cells.

The mice and the strain administration method in Example 1 were used. The bone marrow was collected from the femur of each mouse, and then the distributions of LT-HSCs, short-term HSC (ST-HSCs), and MPP cells were compared by groups using flow cytometry.

As a result, compared with the vehicle group with LT-HSC 35±1.5%, ST-HSC 39±1.5%, and MPP 27±3.0%, the AK group was measured to have LT-HSC 15±2.6%, ST-HSC 43±1.9%, and MPP 41±4.1%, and the AK-P group was measured to have LT-HSC 15±0.7%, ST-HSC 51±3.0%, and MPP 33±3.4%. That is, the aged mice administered with the Akkermansia strain showed significant reductions in LT-HSC and significant increases in MPP (FIG. 6).

It can therefore be seen from the results that the administration of the Akkermansia strain has an effect of inhibiting and ameliorating the aging of hematopoietic stem cells in aged mice.

### Example 8: Analysis of Neutrophil and Lymphocyte Distributions

Linkage skewing, one of the symptoms of aging, is a phenomenon in which the proportion of neutrophils increases and the proportion of lymphocytes deceases in the peripheral blood. A test was performed to investigate the effect of the administration of live and dead Akkermansia strains on neutrophils and lymphocytes in the peripheral blood.

Specifically, aged mice were administered with the Akkermansia strain as described in Example 1, and at 20 weeks of administration, the peripheral blood was collected from each mouse, and then antibody staining was performed with CD45⁺Ly6G⁺CD11b⁺ as a marker for neutrophils and CD45⁺CD3⁺B220⁺ as a marker for lymphocytes, and the distributions of neutrophils and lymphocytes were analyzed using flow cytometry (Table 2).

TABLE 2

| | Neutrophils (%) | Lymphocytes (%) |
|---|---|---|
| Control | 64.8 ± 3.6 | 15.9 ± 2.4 |
| AK group | 47.6 ± 3.6 | 25.1 ± 4.6 |
| AK-P group | 40.5 ± 3.9 | 37.7 ± 4.2 |

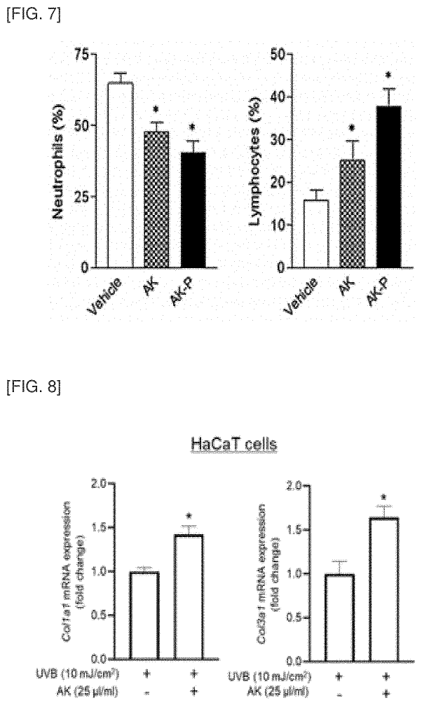

As a test result, the proportion of neutrophils was 64.8±3.6% in the vehicle group, and 47.6±3.6% in the AK group and 40.5±3.9% in the AK-P group, showing significant reductions, and the proportion of the lymphocytes was 15.9±2.4% in the vehicle control group, and 25.1±4.6% in the AK group and 37.7±4.2% in the AK-P group, showing significant increases (FIG. 7).

It was identified from the results that the administration of the Akkermansia strain significantly inhibited and ameliorated linkage skewing, a symptom due to aging, in which the neutrophils increase and the lymphocytes reduce, and it can therefore be seen that the administration of the Akkermansia strain has an effect of treating, inhibiting, and ameliorating aging.

### Example 9: Analysis of Skin Aging Inhibitory Effect

### Example 9-1: Skin Aging Inhibition Test Using Human Keratinocytes

### Example 9-1-1: Cell Culture, Ultraviolet (UVB) Irradiation, and Sample Treatment

The human skin keratinocyte line (HaCaT) was cultured in DMEM containing 10% fetal bovine serum (FBS) and 1% penicillin-streptomycin at 37° C. under a 5% CO₂ condition, and when the cell density reached 80%, the cells were dispensed at 4×10⁵ cells per well in 6-well plates and then cultured for 24 hours. When the fibroblasts cultured in the 6-well plates reached a cell density of 80%, the cells were pre-treated for 1 hour with an AK lysate at a concentration of 25 μL/mL, obtained by culturing at 6.68×10⁹ cells/mL in BTTM and lysis using a sonicator (VCX-500, Sonics, USA) with 20 kHz frequency, 20% amplitude, and operation for 10 seconds and stop for 2 seconds in that order three times. After 1 hour, the cells were washed two times with phosphate buffered saline (PBS), and then irradiated with 10 mJ/cm² ultraviolet light by using an ultraviolet irradiation device (CL-1000 UV crosslinker; UVP, USA). After the UV irradiation, the medium was exchanged with a medium not containing serum, and then immediately, the cells were treated with the same AK sample at a concentration of 25 μL/mL for 22 hours.

### Example 9-1-2: Analysis of Collagen Synthesis Genes (Col1a1 and Col3a1) in Human Skin Keratinocytes

As for skin wrinkles due to skin aging, to investigate the effect of AK on the gene expressions of Col1a1 and Col3a1 as collagen synthetase, the cells were cultured by the above method, and then RNA was isolated from cells in each well by using Trizol (Invitrogen, USA), and quantified with nanodrops, and cDNA was synthesized using 1 μg of RNA for each.

The real-time polymerase chain reaction was performed using a mixture, obtained by adding Col1a1 and Col3a1 primers and AccuPower® 2X GreenStar™ qPCR Master Mix (BIONEER, Korea) as a fluorescent dye to the synthesized cDNA, in the real-time PCR machine, and the gene expression levels of Col1a1 and Col3a1 were analyzed in comparison with that of β-actin (internal control). The test results are shown FIG. 8.

The nucleotide sequences of the primers used in the present test are shown in Table 3.

TABLE 3

| Template | | Sequence |
|---|---|---|
| (primer) | | (5′-3′) |
| Col1a1 | F | GAGGGCCAAG |
| | | ACGAAGACAT |
| | | C |
| | R | CAGATCACGT |
| | | CATCGCACAA |
| | | C |
| Col3a1 | F | GTTTTGCCCC |
| | | GTATTATGGA |
| | R | GGAAGTTCAG |
| | | GATTGCCGTA |
| β-actin | F | GGATTCCTAT |
| | | GTG GGCGA |
| | | CGA |
| | R | CGCTCGGTGA |
| | | GGATCTTCAT |
| | | G |

As a test result, the group treated with the AK lysate, compared with the control group, was observed to show significant increases in the expression of both Col1a1 and Col3a1 (Student's t-test, *p<0.05).

### Example 9-1-3: Analysis of Expression of Collagen Degradation Proteins in Human Skin Keratinocytes

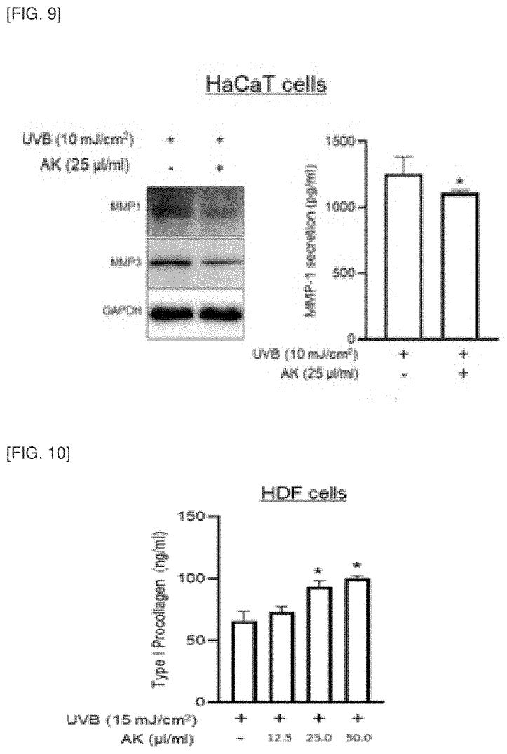

To analyze the protein expressions of the collagenases MMP-1 and MMP-3 by AK treatment on human skin keratinocytes irradiated with ultraviolet light, the cells were cultured by the same method as in Example 9-1-1, and then the proteins were extracted from the cells in each well and subjected to western blot analysis, thereby comparatively analyzing the protein expression levels of MMP-1 and MMP3. In addition, the culture was obtained from each well and the extracellularly secreted MMP-1 protein was quantified using the MMP-1 ELISA kit (Human Total MMP-1 kit, R&D system, USA). The test results are shown FIG. 9.

The photo-aging-induced models obtained by the irradiation of human keratinocytes (HaCaT) with UVB were treated with AK lysate, and the intracellular expressions of MMP-1 and MMP-3 proteins, which degrade collagen proteins, were comparatively analyzed by western blot, and as a result, the AK treatment group was observed to show reductions in the expressions compared with the control group. In addition, as a result of quantitative analysis of MMP-1 secreted into the extracellular culture, the secretion of MMP-1 was significantly inhibited in the group administered with the AK lysate (Student's t-test, *p<0.05).

It was identified that the AK treatment increased the expressions of collagen synthesis genes and significantly inhibited the expressions and secretions of collagenases in the skin aging models induced by UVB on human keratinocytes, and thus it can therefore be seen that the administration of Akkermansia of the present invention has a skin aging inhibitory effect.

### Example 9-2: Skin Aging Inhibition Test Using Human Dermal Fibroblasts

### Example 9-2-1: Cell Culture, Ultraviolet (UVB) Irradiation, and Sample Treatment

The human dermal fibroblasts (Hs68) were cultured in DMEM containing 10% fetal bovine serum (FBS) and 1% penicillin-streptomycin at 37° C. under a 5% CO₂ condition, and when the cell density reached 80%, the cells were dispensed at 4×10⁵ cells per well in 6-well plates and then cultured for 24 hours.

When the fibroblasts cultured in the 6-well plates reached a cell density of 80%, the cells were pre-treated for 1 hour with an AK lysate at concentrations of 12.5 μL/mL, 25 μL/mL, and 50 μL/mL, obtained by culturing at 6.68×10⁹ cells/mL in BTTM and lysis using a sonicator (VCX-500, Sonics, USA) with 20 kHz frequency, 20% amplitude, and operation for 10 seconds and stop for 2 seconds in that order three times. After 1 hour, the cells were washed two times with phosphate buffered saline (PBS), and then irradiated with 15 mJ/cm² ultraviolet light by using an ultraviolet irradiation device (CL-1000 UV crosslinker; UVP, USA). After the UV irradiation, the medium was exchanged with a medium not containing serum, and then immediately, the cells were treated with the same AK sample at concentrations of 12.5 μL/mL, 25 μL/mL, and 50 μL/mL for 26 hours.

### Example 9-2-2: Analysis of Amounts of Secretion of Collagen Synthesis Proteins in Human Fibroblasts

As for skin aging and skin wrinkles, to evaluate the effect of AK on the expression of type I procollagen, the fibroblasts were cultured by the test method as above, and then the cell culture was recovered from each well, and the amount of secretion of type I procollagen was analyzed using the Procollagen Type I C-peptide EIA kit (Takara, Japan). The test results are shown FIG. 10.

The photo-aging-induced models obtained by the irradiation of human fibroblasts with UVB were treated with an AK lysate, and the amount of secretion of type I procollagen associated with skin aging and skin wrinkles was comparatively analyzed. As a result, it was identified that type I procollagen was significantly increased dependent on the AK concentration (Student's t-test, *p<0.05).

It was identified that Akkermansia muciniphila significantly promoted the secretion of type I procollagen in the skin aging models induced by UVB on human dermal keratinocytes, and thus it can therefore be seen that the administration of Akkermansia of the present invention has a skin aging inhibitory effect.

While the present invention has been described with reference to the particular illustrative embodiments, a person skilled in the art to which the present invention pertains can understand that the present invention may be embodied in other specific forms without departing from the technical spirit or essential characteristics thereof. Therefore, the embodiments described above should be construed as being exemplified and not limiting the present invention. The scope of the present invention is not defined by the detailed description as set forth above but by the accompanying claims of the invention, and it should also be understood that all changes or modifications derived from the definitions and scopes of the claims and their equivalents fall within the scope of the invention.

1. A method for preventing or ameliorating aging, the method comprising administering to a subject a composition containing as an active ingredient at least one selected from the group consisting of Akkermansia muciniphila cells, a culture thereof, a lysate thereof, and an extract of the lysate or culture.

2. The method of claim 1, wherein the aging includes at least one selected from the group consisting of muscle aging, skin aging, vision aging, hearing aging, digestive organ aging, immunosenescence, and urinary organ aging.

3. The method of claim 1, wherein the aging is at least one selected from muscle aging and skin aging.

4. The method, wherein the composition is characterized by any one of inhibition of muscular strength weakness, inhibition of hematopoietic stem cell aging, inhibition of immunosenescence, promotion of myoblast differentiation, and inhibition of skin aging.

5. The method of claim 1, wherein the composition further contains a carrier.

6. The method of claim 1, wherein the composition is a pharmaceutical composition, a quasi-drug composition, an animal medicine composition, a health functional food composition, or a feed additive composition.

7. The method of claim 1, wherein Akkermansia muciniphila is contained in the form of live cells, dead cells, or a combination thereof.

8. A method for preventing, treating, or alleviating an aging-related disease, the method comprising administering to a subject a composition containing as an active ingredient at least one selected from the group consisting of Akkermansia muciniphila cells, a culture thereof, a lysate thereof, and an extract of the lysate or culture.
